(19) United States
(12) Patent Application Publication
(10) Pub. No.: US 2012/0089030 A1
(43) Pub. Date: Apr. 12, 2012

(54) SYSTEM AND METHOD FOR CHARACTERIZATION OF ORAL, SYSTEMIC AND MUCOSAL TISSUE UTILIZING RAMAN SPECTROSCOPY

(75) Inventors: Kevin Guze, Boston, MA (US); Michael Short, Coquitlam (CA); Haishan Zeng, Vancouver (CA); Stephen Sonis, Wayland, MA (US)

(73) Assignees: PRESIDENT AND FELLOWS OF HARVARD COLLEGE, Cambridge, MA (US); THE BRIGHAM AND WOMEN'S HOSPITAL, INC., Boston, MA (US); BRITISH COLUMBIA CANCER AGENCY BRANCH, Vancouver, BC (CA)

(21) Appl. No.: 13/144,574

(22) PCT Filed: Jan. 18, 2010
(86) PCT No.: PCT/US10/21315

§ 371 Date: Dec. 29, 2011

Related U.S. Application Data
(60) Provisional application No. 61/145,362, filed on Jan. 16, 2009.

(51) Int. Cl. A61B 6/00

(52) U.S. Cl. 600/476

(57) ABSTRACT

A method and system for characterizing tissue includes a probe connected to a red LASER source and a Raman spectroscope. The probe includes at least excitation fiber and one or more emission fibers that connect the probe with the LASER source and the Raman spectroscope. The excitation fiber is connected to the red LASER source and terminates in the first end of the probe adjacent the tip of the probe. The emission fibers are connected to the Raman spectroscope and terminate in the first end of the probe adjacent the tip of the probe. In one embodiment, the excitation fiber extends through the central portion of the probe and one or more emission fibers are arranged around the excitation fiber. The tip of the probe is intended to come in contact with the tissue to be examined. The tip includes a central opening to allow red LASER radiation to project out of the end of the red excitation fiber on to the tissue and to permit Raman spectra to enter the emission fiber(s) and travel to the Raman spectroscope. The tip is constructed to have a predefined focal length to position the first end of the probe a predefined distance from the surface of the tissue being examined. The tip can be removable and tips having different focal lengths can be used to accommodate different types of tissues and examinations. A detector can convert the Raman spectra into signals and data for analysis by a computer system. The Raman spectra for tissue in a predefined location can be profiled such that the system can distinguish between healthy and diseased tissue.

### CROSS-REFERENCE TO RELATED APPLICATIONS

This application claims any and all benefits as provided by law of U.S. Provisional Application No. 61/145,362 filed Jan. 16, 2009, which is hereby incorporated by reference in its entirety.

### STATEMENT REGARDING FEDERALLY SPONSORED RESEARCH

Not Applicable

### REFERENCE TO MICROFICHE APPENDIX

Not Applicable

### BACKGROUND

1. Technical Field of the Invention

The present invention is directed to methods and systems for characterizing and diagnosing tissue and tissue disease using Raman spectroscopy. Specifically, the invention is directed to a Raman spectrometer system including a Raman spectrometer probe adapted for non-invasive diagnosis of tissue.

2. Description of the Prior Art

In 2008, in the US alone, it was estimated that about 34,000 individuals were to diagnosed with oral cancer. 66% of the time these will be found as late stage three and four disease. Low public awareness of the disease is a significant factor, but these cancers could be found at early highly survivable stages through from examination by a trained medical or dental professional.

Although oral cancer is the most serious of oral cavity disease, and is often life threatening, it makes up only a small fraction of the total number of oral diseases. However benign oral diseases can also be severe and debilitating if not treated properly and at an early stage.

Histology has been the gold standard for diagnosing the overwhelming majority of oral mucosal diseases including malignancies and autoimmune conditions. Despite its desirability as a means to provide a definitive diagnosis, logistical, psychological, and economic hurdles often negatively impact on the frequency with which biopsies are performed. Consequently, there has been increasing interest to develop alternative means for diagnosis including cytological techniques, the use of cell markers, and the application of optical coherence imaging technology. In vivo Raman measurements are particularly challenging to acquire since the spectra must be obtained with a short integration time, and often require the use of optical fibers which introduce significant noise into the spectra. This noise is considerably reduced by choosing ultra low OH fiber; nevertheless it remains a problem in the fingerprint region (400-1800 cm⁻¹). This has prompted some investigators to look at the high frequency (HF) region (1800-3500 cm⁻¹) of the spectra. Although there are fewer Raman peaks in the HF region, they had considerable success in using the C-H stretch bands near 3000 cm⁻¹ to discriminate between different tissue types.

What is needed is a combined Raman and fluorescence oral analyzer, as well as fluorescence observation which can be used to identify abnormal tissue areas (benign lesions and cancers), along with Raman spectroscopy measurements using the same system to differentiate cancer from benign lesions.

### SUMMARY

The present invention is directed to a Raman spectrograph system for measuring Raman spectra of tissue. The system includes a Raman spectrograph probe having an elongated handle extending from a first end to a second end and a contact tip extending a predefined distance from the first end. The system includes a first laser source adapted to produce a first laser radiation at a first predefined wavelength directed at the tissue and a first excitation fiber coupled to the laser source and extending up to the first end of the Raman spectrograph probe and adapted to transfer laser radiation to the first end. The system further includes a plurality of emission fibers coupled to the Raman spectrograph and extending up to first end of the Raman spectrograph probe and adapted to transfer Raman spectra received from the tissue at the first end of the Raman spectrograph probe to the Raman spectrograph. The system includes a Raman spectrograph for generating Raman spectra signals and a detector for producing Raman spectra data from the Raman spectra signals.

The tip of the probe extends from the first end of the probe and positions the first end of the probe a predefined distance from the surface of the tissue to be examined, defining the focal length of the system. The tip can be removable and disposable or cleaned by washing or autoclaving. The tip includes a central opening that permits an excitation laser to project from the end of the excitation fiber at the first end onto the tissue to be examined and Raman spectra generated by the tissue as a result of the projected laser radiation can be received at the end of one or more emission fibers in the first end of the probe and transmitted to the Raman spectrograph. The Raman spectrograph and the detector can generate Raman spectra data that is characteristic of the tissue being examined. Filters can be used to block unwanted signals and noise. From the Raman spectra data, Raman spectra profiles of healthy and diseased tissue can be determined and used to diagnose tissue without biopsy.

The system according to the invention can be used to characterize tissue by generating Raman spectra profiles that can include signals indicative of the principal components of the tissue. The probe tip is placed in contact with the tissue and the first laser is energized or activated causing the laser radiation to illuminate the tissue. The tissue produces Raman spectra in response to the laser radiation and the Raman spectra can be transferred to the Raman spectrograph and associated detector which produce data signals representative of the Raman spectra. The data signals can be stored in a computer and processed to produce tissue profiles or fingerprints that can be used to distinguish between tissue having different molecular components, such as healthy tissue and diseased tissue.

In accordance with the invention, the probe can include a second laser radiation source that can be projected from the first end of the probe. The wavelength of the second laser radiation source can produce radiation that is known to cause diseased tissue to fluoresce and be visible with the use of a filter. The second laser radiation can be used to illuminate an area to identify potentially diseased tissue and then using the Raman system according to the invention, capture Raman spectra of the tissue, compare the Raman spectra of the potentially diseased tissue with the Raman spectra of healthy tissue to determine whether the tissue is diseased. This can be accomplished by producing a Raman spectra profile or fingerprint of the potentially diseased tissue and comparing the profile or fingerprint to those of known good tissue and/or known diseased tissue, assessing similarities and/or differences in order to assist diagnosis.

One of the advantages of the present invention is that it provides a fast and non-invasive analysis of potentially diseased tissue.

Another advantage of the present invention is that it can be used in a clinical setting.

A further advantage of the present invention is that can be used to diagnose diseased tissue at an earlier stage of the disease and increase the likelihood of successful treatment.

### BRIEF DESCRIPTION OF THE FIGURES

The accompanying drawings, which are incorporated into and constitute a part of this specification, illustrate one or more examples of embodiments and, together with the description of example embodiments, serve to explain the principles and implementations of the embodiments.

FIG. 1 is a diagrammatic view of a system according to the invention.

FIGS. 2A and 2B show diagrammatic views of embodiments of a probe tip according to the invention shown in FIG. 1.

FIGS. 3-4 show diagrammatic views of cross-sections of the cable according to the invention.

FIG. 5 shows a comparison of average spectra data from different oral tissue sites obtained according to the invention.

FIGS. 6A and 6B show graphs of normalized intensity values for different oral tissue sites as a function of wavenumber from the study.

FIGS. 7A and 7B show graphs of Engenvalues as a function of factor number from the study.

FIGS. 8A, 8B, and 8C show graphs of Factor score as a function of spectrum number from the study.

FIG. 9 shows a table that illustrates the classification by race of oral Raman Spectra using LDA according to the study.

FIG. 10 shows a table that illustrates classification by oral tissue side of oral Raman Spectra using LDA according to the study.

### DETAILED DESCRIPTION OF PREFERRED EMBODIMENTS

Example embodiments are described herein in the context of a system and method for characterization of tissue utilizing Raman Spectroscopy. Those of ordinary skill in the art will realize that the following description is illustrative only and is not intended to be in any way limiting. Other embodiments will readily suggest themselves to such skilled persons having the benefit of this disclosure. Reference will now be made in detail to implementations of the example embodiments as illustrated in the accompanying drawings. The same reference indicators will be used throughout the drawings and the following description to refer to the same or like items.

In the interest of clarity, not all of the routine features of the implementations described herein are shown and described. It will, of course, be appreciated that in the development of any such actual implementation, numerous implementation-specific decisions must be made in order to achieve the developer's specific goals, such as compliance with application- and business-related constraints, and that these specific goals will vary from one implementation to another and from one developer to another. Moreover, it will be appreciated that such a development effort might be complex and time-consuming, but would nevertheless be a routine undertaking of engineering for those of ordinary skill in the art having the benefit of this disclosure.

In accordance with this disclosure, the components, process steps, and/or data structures described herein may be implemented using various types of operating systems, computing platforms, computer programs, and/or general purpose machines. In addition, those of ordinary skill in the art will recognize that devices of a less general purpose nature, such as hardwired devices, field programmable gate arrays (FPGAs), application specific integrated circuits (ASICs), or the like, may also be used without departing from the scope and spirit of the inventive concepts disclosed herein. It is understood that the phrase “an embodiment” encompasses more than one embodiment and is thus not limited to only one embodiment. Where a method comprising a series of process steps is implemented by a computer or a machine and those process steps can be stored as a series of instructions readable by the machine, they may be stored on a tangible medium such as a computer memory device (e.g., ROM (Read Only Memory), PROM (Programmable Read Only Memory), EEPROM (Electrically Eraseable Programmable Read Only Memory), FLASH Memory, Jump Drive, and the like), magnetic storage medium (e.g., tape, magnetic disk drive, and the like), optical storage medium (e.g., CD-ROM, DVD-ROM, paper card, paper tape and the like) and other types of program memory.

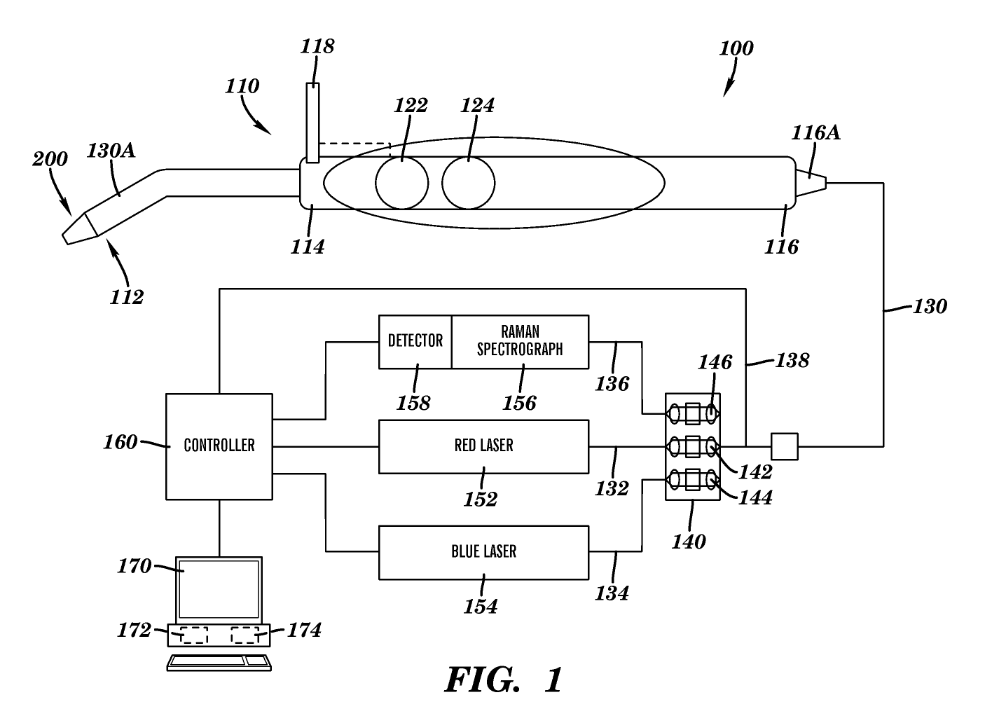

FIG. 1 shows a Raman spectroscopy system 100 in accordance with an embodiment of the present invention. The system 100 includes a probe 110, cable 130, a filter module 140, a red LASER 152, a blue LASER 154, a Raman spectrograph 156, a detector 158, a controller 160 and a computer 170. The probe 110 includes an examination tip 200 at a first end 112 of the probe 110 extending from a protective sheath 130A that extends from an elongated handle 114 and the cable 130 extending from the second end 116 of the probe 110. In some embodiments of the invention, the probe 110 can also include a long pass filter shield 118 for viewing tissue fluorescence, for example, a long pass filter having a cutoff above the wavelength of the blue LASER 154 to allow the red or green tissue fluorescence to be viewed. The filter shield 118 can be removable or be the fold up/down or pop-up/down type shield so it can be removed from view as necessary. The probe 110 can also include controls for controlling the operation of the system, including a trigger button or switch 122 and an excitation button or switch 124. The trigger button or switch 122 can be connected to the control cable 138 and configured to open or close a circuit to trigger the operation of the system to activate the detector 158 to detect Raman spectra produced by the Raman Spectrograph 156. The excitation button or switch 124 can be connected to the control cable 138 and configured to open or close a circuit to cause one or more of the excitation sources (e.g., red LASER 152 or blue LASER 154) to turn on or off. In one embodiment of the invention, when the excitation button is not pressed, the red LASER 152 is on (optionally at less than full power), illuminating the red excitation fiber 132 and the blue LASER 154 is off, and when the excitation button is pressed, the red LASER 152 is turned off, the blue LASER 154 is turned on, illuminating the blue excitation fiber 134. In accordance with one embodiment of the invention, when red LASER 152 is activated according to the state of the excitation button or switch 124, the red LASER 152 can be operating at less than full output power, for example less than 75% or less than 50% or less than 25% of full output power and when the trigger button or switch 122 is activated, the red LASER 152 can be activated to full or a higher percentage of maximum output power. In accordance with one embodiment of the invention, the red LASER 152 is energized to 10% of full output when turned on (such as by the release of the excitation button 124) and is energized to 100% full power when the trigger button 122 is activated.

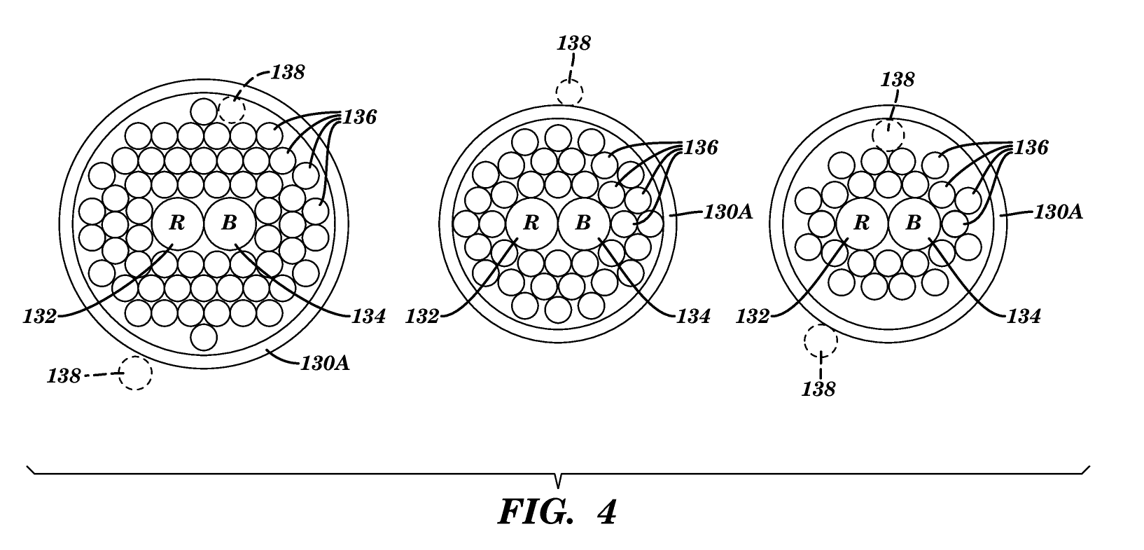

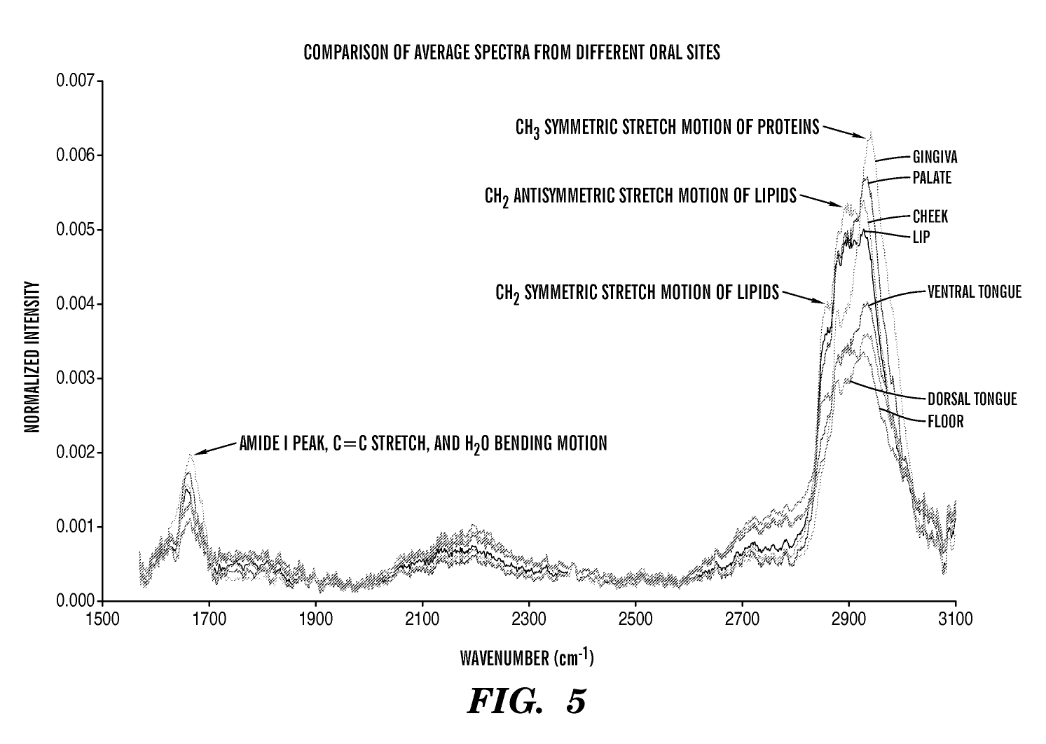

The cable 130 can extend through a strain relief component 116A at the second end of the probe handle 114 and extend several feet to the filter module 140. The cable 130 can include one or more excitation fibers, such as a red excitation fiber 132 which can be connected to a red LASER 152 and a blue excitation fiber 134 which can be connected to a blue LASER 152. The cable 130 can also include a plurality of emission fibers 136 which can be connected to the Raman spectrograph 156. The cable 130 can also include the control cable 138 which can be connected to the controller 160. In accordance with one embodiment of the invention, the excitation fibers 132 and 134 can be high performance fiber optic cables that provide very low signal loss in the wavelength of the optical signal being transferred. In accordance with one embodiment of the invention, each of the excitation fibers 132 and 134 can be 100-200 micrometer low or ultra low OH fiber optic cable and the emission fibers 136 can be 50-100 micrometer low or ultra low OH fiber optic cable. The emission fibers 136 can be bundled around the concentrically located excitation fiber(s) 132 and 134 in various configurations as shown in FIGS. 4 and 5, having an approximate diameter of 1.8 millimeters. The fiber bundle including the excitation fibers 132 and 134 and the emission fibers 136 as well as the control cable 138 can be enclosed or encased in a protective sheath to prevent unwanted noise from entering the fibers and protect them from wear.

The cable 130 can be, for example 0.75 meters long and can be configured to include filters at the proximal or first end 112 in the probe 110 and the distal end which is connected to the filter module 140. The filters can include band pass filters at the ends of the excitation fibers 132 and 134 and selected to pass a specific wavelength of light that needs to be carried through the fiber. The filters can also include long pass filters, connected to the emission fibers 136, selected to block signals below a selected cutoff wavelength. The individual optical fibers can include sheathing and/or cladding that minimize or eliminate cross talk, the transfer signals between adjacent optical fibers within cable 130. The purpose of the filters and cladding is to reduce or eliminate this noise from being transferred to the tip 200 of the probe 110 through the excitation fibers 132 and 134 and to the Raman spectrograph 156 through the emission fibers 136.

In accordance with one embodiment, the cable 130 can include a filter module 140 connected between the probe 110 and the red LASER 152, the blue LASER 154 and the Raman spectrograph 156. The filter module 140 can include separate, high performance filters connected to each optical fiber in the cable 130. The filter module 140 can include a band pass filter 142 connected inline in the red excitation fiber 132 which is selected to pass only the wavelength corresponding to the light output by the red LASER 152 and block the background Raman and fluorescence signals generated inside the red excitation fiber 132. The filter module can include a band pass filter 144 connected inline in the blue excitation fiber 134 which is selected to pass only the wavelength corresponding to the light output by the blue LASER 154 and block the background Raman and fluorescence signals generated inside the blue excitation fiber 134. The filter module 140 can also include along pass filter 146 connected inline in the emission fibers which is selected to pass the Raman spectra signals above a selected cutoff wavelength and block the background Raman and fluorescence signals generated inside the emission fibers. The Raman signals can be refocused by the filter module 146 into the round-to-parabolic linear array emission fiber bundle 136 as described in U.S. Pat. No. 6,486,948 and No. 7,383,077 which are hereby incorporated by reference in their entirety.

In accordance with one embodiment of the invention, the system 100 can include a red LASER 152 connected to excitation fiber 132 to transmit the red LASER radiation to the tip 200 of the probe 110. The wavelength of the red LASER 152 can be selected from the red, near infrared and infrared ranges to optimally provide the desired Raman spectra response for the tissue being examined. The wavelength of the red LASER 152 can, for example, be selected to provide red LASER radiation having a wavelength in the range from 700 to 850 nanometers. In one embodiment, the wavelength of red LASER 152 can be selected to provide red LASER radiation having a wavelength in the range from 760 to 840 nanometers and an output power in the range from 100 to 350 mW. In one embodiment, the red LASER 152 provides red LASER radiation having a wavelength of 785 nanometers using a 300 mW temperature stabilized diode LASER (from B&W Tek, Newark, Del., model; BRM 785). This wavelength has been found to provide good results for mucosal tissue. The output power can be selected as function of the desired system performance. The maximum output power of the red LASER can be limited to a safe margin below the point at which the LASER can cause damage to the tissue being examined. However, the lower the output power of the red LASER, the lower the energy of the Raman spectra, making it difficult to detect and requiring longer detection times. The output power of the red LASER can be selected to provide acceptable detection times without causing damage to the tissue being examined.

In accordance with one embodiment of the invention, the system 100 can include a blue LASER 154 connected to excitation fiber 134 to transmit the blue LASER radiation to the tip 200 of the probe 110. The wavelength of the blue LASER 154 can be selected to optimally provide the desired fluorescence for the tissue being examined. It is known that tissue that emits fluorescence when exposed to this blue LASER radiation can be characterized as diseased tissue. The wavelength of the blue LASER 154 can, for example, be selected to provide blue LASER radiation having a wavelength in the range from 400 to 460 nanometers and an output power of 50 mW to 300 mW. In one embodiment, the blue LASER 154 provides blue LASER radiation having a wavelength of 430 nanometers and an output power of 100 mW. This wavelength has been found to provide good results for mucosal tissue. The output power of the blue LASER can be selected to achieve the desired function of causing diseased to fluoresce without causing damage to the tissue being examined.

In accordance with one embodiment of the invention, the system 100 can include a Raman spectrograph 156 connected to a detector 158. The Raman spectrograph 156 can be connected to the emission fibers 136 to enable Raman spectra received from the irradiated tissue to be transmitted to the Raman spectrograph 156 for presentation to the detector 158 to produce Raman spectra data. The detector 158 can be a charged coupled device (CCD) based sensor that quantizes and outputs the spectral data as an array of intensities at different wavelengths or wavenumbers. In one embodiment, the Raman spectrograph included a Holospec f/2.2 transmissive imaging spectrograph, available from Kaiser Optical Systems of Ann Arbor, Mich. and the detector was a Spec-10:400 BR/LN liquid nitrogen cooled CCD array having 400×1340 pixels@ 20×20 micrometers per pixel, available from Princeton Instruments, Trenton, N.J. In addition, a parabolic array configuration can be used so that all the light at a particular wavenumber that is collected from the sample can be projected onto the CCD detector in a straight line providing an improved signal to noise ratio.

In accordance with one embodiment of the invention, the system 100 can include a controller 160 which can provide an interface for connecting the various components of the system to a computer system 170, such as an Apple Macintosh or a Linux or Microsoft Windows based personal computer. The controller 160 can be adapted and configured to control the power to the red LASER 152 (e.g., using a power transformer or a relay) to turn the LASER on and off as well as to control the output power of the LASER using a serial or parallel interface control signals. Alternatively, the red LASER 152 can be self powered and only controlled through controller 160 as described herein or using a wired interface, such as Universal Serial Bus (USB), Firewire, serial (RS232) or parallel interface or a wireless interface, such as Wi-Fi, Blue Tooth, or ZigBee. The controller 160 can be adapted and configured to control the power to the blue LASER 154 (e.g., using a power transformer or a relay) to turn the LASER on and off as well as to control the output power of the LASER using a serial or parallel interface control signals. Alternatively, the blue LASER 154 can be self powered and controlled through controller 160 as described herein or using a wired interface, such as Universal Serial Bus (USB), Firewire, serial (RS232) or parallel interface or a wireless interface, such as Wi-Fi, Blue Tooth, or ZigBee. The controller 160 can be adapted and configured to control the power to the detector 158 and Raman spectrograph 156 (e.g., using a power transformer or a relay) to turn the detector on and off and to read Raman spectra data as well as to receive the spectra data signals from the detector using a serial or parallel interface. Alternatively, Raman spectrograph 156 and the detector 158 can be self powered and controlled by the controller 160 and send Raman spectra data to the controller as described herein or using a wired interface, such as Universal Serial Bus (USB), Firewire, serial (RS232) or parallel interface or a wireless interface, such as Wi-Fi, Blue Tooth, or ZigBee. The controller 160 can received Raman spectra data from the detector 158 and forward it to the computer system 170 for further processing and analysis. In addition, the controller 160 can receive control signals from the computer system 170 to control the operation of the red LASER 152, the blue LASER 154, the Raman spectrograph 156 and/or the detector 158. In addition, the controller 160 can be connected to the trigger button or switch 122 to allow operation of the trigger button or switch 122 to enable to the Raman spectrograph 156 and detector 158 to take Raman spectra reading upon the pressing or depressing of the trigger button or switch 122. In some embodiments of the invention, the operation of the trigger button or switch 122 can be processed and controlled by the computer system 170 with the computer system 170 sending the control signals to Raman spectrograph 156 and detector 158 to start and stop the generation of Raman spectra data. The controller 160 can be connected to the excitation button or switch 124 to allow operation of the excitation button or switch 122 to turn the LASERS 152 and 154 on and off upon the pressing or depressing of the excitation button or switch 122. In accordance with one embodiment of the invention, pressing the excitation button or switch 124 can cause one LASER (e.g., blue LASER 154) to turn on and the other LASER (e.g., red LASER 152) to turn off and releasing or depressing the excitation button or switch 124 can cause one LASER (e.g., red LASER 152) to turn on and the other LASER (e.g., blue LASER 154) to turn off.

The controller 160 can be a dedicated device based upon an application specific integrated circuit (ASIC), programmable array or programmable micro controller. Alternatively, the controller 160 can be an interface which controls and converts signals for transfer between the components of the system and the computer system 170. The controller can include analog to digital conversion functions to convert Raman spectra signals from the detector 158 to digital data signals transferred to the computer system 170.

The computer system 170 can include a CPU or processor 172 and associated memory 174, including RAM, ROM, volatile and non-volatile memory for storing and executing programs and storing data. The computer system 170 can include programs for reading in, storing and displaying Raman spectra data received from the detector 158, performing analysis and processing of the Raman spectra data and for comparing the received Raman spectra data with stored Raman spectra data. The Raman spectra data can be displayed in the form of graphs and tables.

In an alternative embodiment of the invention, the system 100 can combine the utility of the oral mucosal tissue green/ted fluorescence excited by the blue LASER 154 with Raman spectroscopy for diagnosing malignant and pre-malignant tissue. The system 100 can include a blue LASER 154 coupled to the controller 160, whereby the blue LASER 154 is in communication with the filter module 140. The combined blue and red light can be transmitted through a single excitation fiber 132 for fluorescence excitation and Raman excitation of the mucosal tissue.

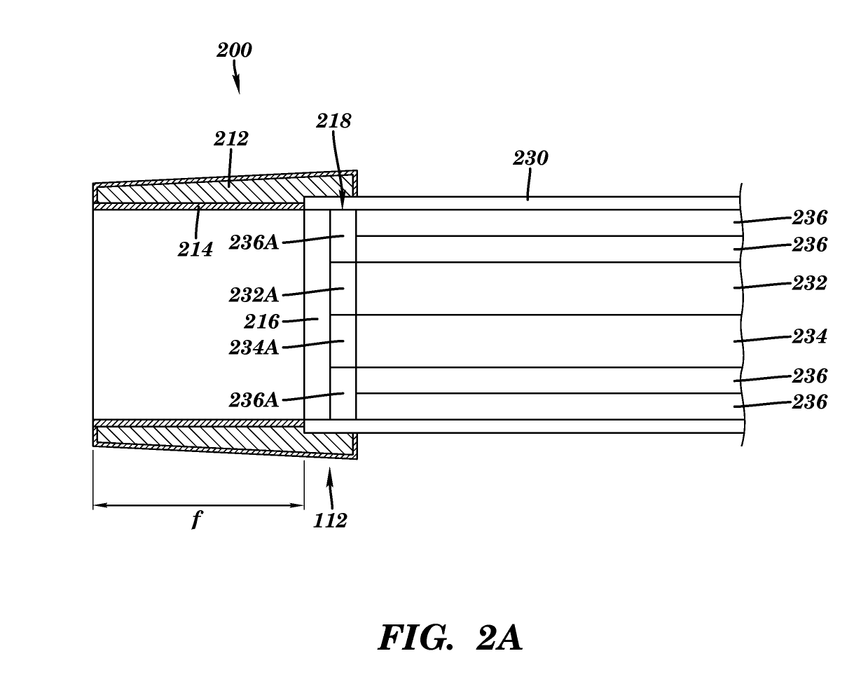

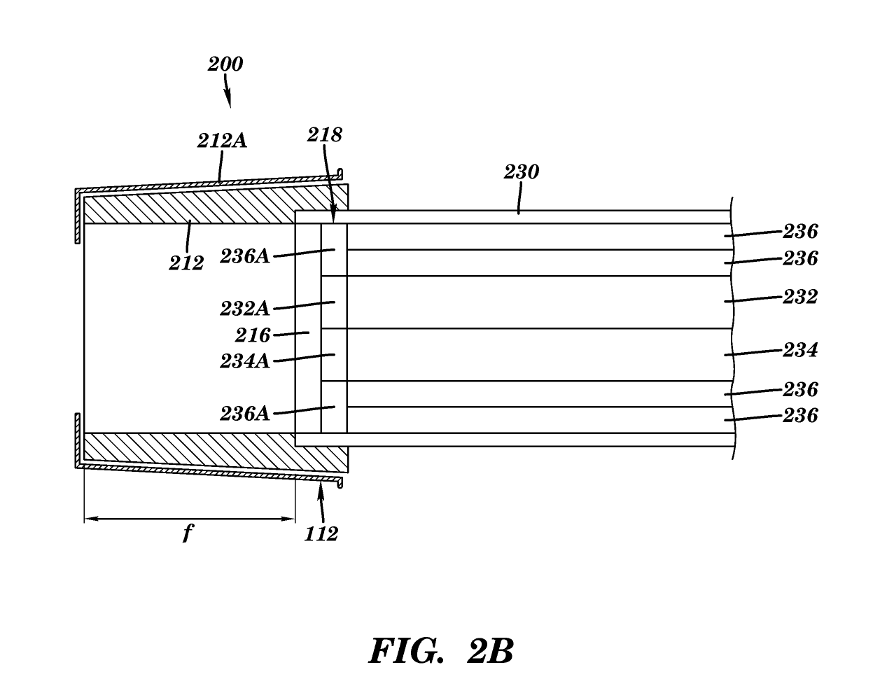

FIGS. 2A and 2B show alternative configurations of the tip 200 at the first end 112 of the probe 110. In accordance with one embodiment of the invention, the first end 112 of the probe 110 can include a protective cover 216 and one or more filters 218 adjacent to the first ends of the excitation fibers 232 and 234 and the emission fibers 236. As shown in FIGS. 2A and 2B, the excitation fibers 232 and 234 and the emission fibers 236 can be enclosed in protective sheath 230, 130A, such as stainless steel or titanium tubing extending from the probe handle 114 to protect the fibers from damage and assist the operator in positioning the tip 200 on the first end 112 of the probe 110 in contact with the tissue to be analyzed. As shown in FIG. 1, the protective sheath 230, 130A can include one or more bends to facilitate insertion and contact with mucosal or other tissue.

In accordance with one embodiment, the end of each excitation fiber 232 and 234 and the end of each emission fiber 236 can include a filter 232A, 234A and 236A to reduce noise in the system. For each excitation fiber 232 and 234, the first end 112 can include a band pass filter 232A and 234A selected to pass only the wavelength of the excitation LASER radiation and block Raman emissions generated in the fiber. The filter 232A and 234A can be a separate material, such as glass or quartz, positioned adjacent or affixed to the end of the excitation fiber or the filter 232A and 234A can be a coating applied to the end of the fiber. For each emission fiber, the first end 112 can include a long pass filter 236A selected to pass only wavelengths above the cutoff wavelength that correspond to the Raman spectra to be measured and block the LASER wavelengths. The filter 236A can be a separate material, such as glass or quartz, positioned adjacent or affixed to the end of each emission fiber or the filter 236A can be a coating applied to the end of each emission fiber. In accordance with the invention, for the red excitation fiber 232, the filter 232A can be in the range of 700 to 850 nanometers and preferably in the range of 760 to 840 nanometers. In one embodiment, for the red excitation fiber 232, the filter 232A can be a 785 nanometer filter that takes the form of a coating applied to the polished end of the red excitation fiber 232. In accordance with the invention, for the blue excitation fiber 234, the filter 234A can be in the range of 400 to 460 nanometers. In one embodiment, for the blue excitation fiber 234, the filter 234A can be a 430 nanometer filter that takes the form of a coating applied to the polished end of the blue excitation fiber 234. In accordance with the invention, for each emission fiber 236, the filter 236A can be a long pass filter having a cutoff in the range of 800 to 860 nanometers and preferably in the range of 820 to 850 nanometers. In one embodiment, for each emission fiber 236, the filter 236A can be an 830 nanometer long pass filter that takes the form of a coating applied to the polished end of each emission fiber 236. In accordance with an alternative embodiment of the invention, the filter 218 can be a concentric filter formed of a glass or quartz material having the band pass filters 232A and 234A in the center and the long pass filter 236A around the outer portion of the concentric filter. In this embodiment, the ends of the excitation 232 and 234 and emission 236 fibers can be positioned adjacent to or up against the filter 218 as shown in FIG. 2A.

In accordance with one embodiment of the invention, the first end 112 of the probe 110 can include a quartz protective cover 216 which protects the filters at the end of each of the excitation 232 and 234 and the emission 236 fibers. The protective cover can, for example, be a hardened glass or quartz plate held in place by the protective sheath 230, 130A.

In accordance with the invention a tip 200 can be removably attached to the first end 112 of the probe 110 to position the first end 112 a predefined distance or focal length, f, from the tissue being examined. The tip 200 can include an opening that allows the excitation radiation emanating from the red excitation fiber 232 and the blue excitation fiber 234 to be projected onto the tissue being examined. In accordance with the invention, the tip 200 can position the first end 112 of the probe 110 in the range of 3 to 10 mm from the tissue being examined. In accordance with one embodiment of the invention, the tip 200 can provide a focal length in the range of 5-7 mm. In accordance with one embodiment of the invention the tip 200 provides a focal length of 6 mm. In accordance with other embodiments of the invention, a kit of tips of the same or different lengths can be provided, where each tip 200 in the kit provides a predefined focal length in the range from 3 to 10 mm and the LASERS are tunable over a range of wavelengths. In this embodiment, the filters 218 and 140 can be removable and different filters 218, 232A, 2328, 236A can be inserted in the first end 112 and different filter modules 140 or individual filter elements 142, 144, 146 can be inserted to accommodate different excitation wavelengths and Raman spectra wavelengths.

In accordance with one embodiment of the invention, as shown in FIG. 2A, the tip 200 can be removable from the first end 112 of the probe 100 and either disposable or capable of being cleaned by washing or autoclaving, in order to be reused. The tip 200 can be made of a metal, ceramic, glass or plastic material 212 with a central opening that slides or snaps onto the first end 112 of the probe 110. The tip 200 can be opaque to prevent outside light from penetrating the tip, have extremely low (or no) auto-fluorescence when exposed to the excitation LASER radiation used by the system 100 and extremely low (or no) Raman emission when exposed to the excitation LASER radiation used by the system 100. Alternatively, the tip 200 can include a coating or sleeve 214 on the inner surface that provides some or all of these desired properties. In accordance with one embodiment of the invention, the tip 200 can be formed from a Teflon™ material, with or without a coating or sleeve on the inner surface. Alternatively, the tip 200 can be formed from a Pyrex™ (or other toughened glass) material and coated on the inner surface to provide a reusable tip that can be washed or autoclaved between uses. The coating used can be a short pass filter coating similar that used on excitation fibers 132 and 134, which allows all scattered LASER light (for example, at 785 nm) to pass through while reflecting longer Raman wavelengths. This short pass coating prevents Raman emissions from escaping through the tip and blocks ambient room light in the measured Raman wavelengths. This coating can be a short pass coating that is available from Chroma Technology Corp., Rockingham, Vt. and Semrock, Inc., Rochester, N.Y.

In accordance with one embodiment of the invention, as shown in FIG. 2B, the tip 200 can be made of removable from the first end 112 of the probe 100 and be provided with a disposable protective cover. The tip 200 can be made of a metal, ceramic, glass or plastic material 212 with a central opening that slides or snaps onto the first end 112 of the probe 110 and a protective rubber or plastic or paper cover. 212A that fits over the tip 200 can be provided to protect the tip 200 and prevent the spread of infection or disease. In one embodiment, the protective cover can have a hole that is smaller than the central opening in the tip 200. The tip 200 and/or the protective cover 212A can be opaque to prevent outside light from penetrating the tip, have extremely low (or no) auto-fluorescence when exposed to the excitation LASER radiation used by the system 100 and extremely low (or no) Raman emission when exposed to the excitation LASER radiation used by the system 100. Alternatively, the tip 200 can include a coating or sleeve 214 (as shown in FIG. 2A) on the inner surface that provides one or more of these desired properties. In accordance with one embodiment of the invention, the tip 200 can be formed from a Teflon™ material, with or without a coating or sleeve on the inner surface. Alternatively, the tip 200 can be formed from a Pyrex™ (or other toughened glass) material and coated on the inner surface to provide a cleanable and reusable tip. The coating used can be a coating similar that used on excitation fibers 132 and 134, which allows all scattered LASER light (for example, at 785 nm) to pass through while reflecting longer Raman wavelengths. In addition, probe 110 can be configured to provide a high signal to noise ration as described in U.S. Pat. No. 6,486,948 and No. 7,383,077.

In accordance with one embodiment of the invention, the blue LASER 154, the excitation button 124, the filter 118 and the blue excitation fiber 134 can be omitted from the system 100. In accordance with this embodiment, the system 100 can be used by a technician, a nurse or a physician trained in its operation. In accordance with the invention, the system 100 can be used to produce Raman spectra data and profiles for various forms of healthy and diseased tissue (including malignant and pre-malignant tissue), including mucosal tissue. The user can turn the system on and point the tip of the probe at the tissue, to be examined. Upon identifying an area of tissue to be examined and profiled, the user can place the tip 200 of the probe 110 in contact with the surface of the tissue and press the trigger button 122. When the user presses the trigger button 122, the system begins to measure the Raman spectra emitted from the tissue being examined. The user can press the trigger button 120 for one second (or any predefined length of time) or the system, using the controller 160 or computer system 170, can control the process of measuring the Raman spectra for a predefined or preprogrammed period of time. For each area of tissue examined, the system 100 can record, in the computer system 170, the Raman spectra data as well as a profile or fingerprint of the Raman spectra. The system 100 can store profiles of Raman spectra for normal tissue and compare the Raman profiles of tissue being examined with Raman profiles for normal tissue to enable a user to determine whether the differences indicate disease, such as cancer.

In accordance with the invention, the system 100 can be used by a technician, a nurse or a physician trained in its operation. In accordance with one embodiment of the invention, the system 100 can be used to detect diseased, cancerous and pre-cancerous tissue, including mucosal tissue. The user can turn the system on and point the tip of the probe at the tissue, to be examined. The user can press the excitation button 124 to turn on the blue LASER 154 causing blue LASER radiation to project from the tip 200 onto the tissue to be examined. The blue LASER radiation at the wavelength of 430 nanometers can cause areas of diseased tissue to fluoresce red and green and this red/green fluorescence can be made visible to the user when viewed through the filter 118. Alternatively, the red/green fluorescence can be observed using appropriate filter goggles. Upon identifying an area of diseased tissue that emits fluorescence, the user can place the tip 200 of the probe 110 in contact with the surface of the area and release the excitation button 124. Releasing excitation button 124 can cause the blue LASER 154 to turn off and the red LASER 152 to turn, on (optionally, not at full power). The red LASER radiation will be projected onto the diseased tissue causing Raman spectra to be generated. The user can then press the trigger button 122 to cause (optionally, the red LASER 152 to energize to full power and) the system to measure the Raman spectra emitted from the suspected area of diseased tissue being examined. The Raman spectra data can be transferred from the detector 158 through the controller 160 to the computer system 170. A Raman spectra profile for the tissue being examined can be compared with healthy tissue profiles and/or known disease profiles and based upon preprogrammed threshold differences and/or similarities, provide an indication of whether the tissue being examined is diseased and if so, potential disease types, such as cancer.

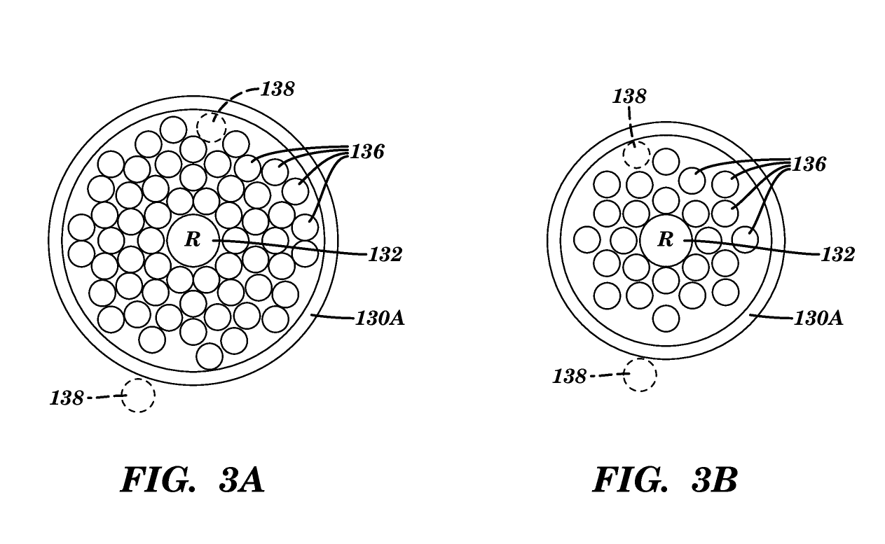

In accordance with the invention, the excitation fibers 132 and 134 and the emission fibers 136 can be arranged in bundles that are round, oval, rectangular, square or any other shape. FIGS. 3A and 3B show configurations having a single red excitation fiber 132 and a plurality of emission fibers 136 in accordance with the invention. FIG. 3A shows one configuration in accordance with one embodiment of the invention wherein 54 emission fibers 136 are arranged in a round or circular configuration around a red excitation fiber 132. The control cable 138 could be included inside the outer protective sheath 230, 130A or control cable 138 can be tied, for example using cable ties, to the outside of the sheath. FIG. 3B shows one configuration in accordance with one embodiment of the invention wherein 24 emission fibers 136 are arranged in a round or circular configuration around a red excitation fiber 132. The control cable 138 could be included inside the outer protective sheath 230, 130A or control cable 138 can be tied, for example using cable ties, to the outside of the sheath.

FIGS. 4A, 4B and CB show configurations having a red excitation fiber 132, a blue excitation fiber 134 and a plurality of emission fibers 136 in accordance with the invention. FIG. 4A shows one configuration in accordance with one embodiment of the invention wherein 54 emission fibers 136 are arranged in a round or circular configuration around a red excitation fiber 132 and a blue excitation fiber 134. The control cable 138 could be included inside the outer protective sheath 230, 130A or control cable 138 can be tied, for example using cable ties, to the outside of the sheath. FIG. 4B shows one configuration in accordance with one embodiment of the invention wherein 36 emission fibers 136 are arranged in a round or circular configuration around a red excitation fiber 132 and a blue excitation fiber 134. The control cable 138 could be included inside the outer protective sheath 230, 130A or control cable 138 can be tied, for example using cable ties, to the outside of the sheath. FIG. 4C shows one configuration in accordance with one embodiment of the invention wherein 24 emission fibers 136 are arranged in a round or circular configuration around a red excitation fiber 132 and a blue excitation fiber 134. The control cable 138 could be included inside the outer protective sheath 230, 130A or control cable 138 can be tied, for example using cable ties, to the outside of the sheath.

In accordance with an alternative embodiment of the invention, the probe 110 can include a red LASER diode alone or a red LASER diode and a blue LASER diode in the handle 114 and a appropriate power source to power the diodes to generate the LASER radiation and feed it into the tip using a short length of excitation optical fiber without the need for a cable 130. Further, a short length of emission optical fibers can be coupled to optical sensors that produce electrical signals that can be transmitted wirelessly to a Raman spectrograph 156 and associated detector 158 to produce Raman spectra data.

The system according to the invention, using Raman spectroscopy can be used as an optical biopsy. For example, the physician may have already identified areas of interest using other modalities, such as white light, and/or fluorescence imaging. For some benign conditions, the patient may be already experiencing some symptoms, and the affects of the disease can be seen in the tissue with morphology and/or color changes under different illumination conditions. Once these tissue areas are identified, Raman spectra can be obtained from them using the present invention.

In accordance with one embodiment, the Raman probe is used to obtain measurements, by holding or positioning the probe 5 to 10 mm from each designated site for one second. It should be noted that other distances from the tissue can be used and other durations of time in obtaining Raman spectra measurements can be used. RS Spectra from the one or more designated oral tissue sites within the patient's mouth can be recorded and saved for later comparison or analysis. Such sites may include, but are not limited to, movable buccal mucosa, attached gingiva, dorsal surface of the tongue, ventral surface of the tongue, the floor of the mouth, the movable mucosa of the lower lip, and the hard palate.

The Raman signals received by the system 100 can include several data values or characteristics which can be used by the system 100 to identify and/or classify the tissue being examined or diagnosed. In an embodiment of the invention, the system 100 can be used to sample only one specimen of oral tissue in vivo or ex vivo, although more than one sample (such as a different oral tissue site or same oral tissue site in another patient) may be taken in vivo (or ex vivo) and then analyzed. For example, oral tissue samples of two or more patients may be taken and compared using the system to determine molecular differences in the tissue among different genders and/or races. In another example, analyzed data from the system of prior sampled tissues may be stored in a local or central database to be retrieved to allow researchers to compare healthy oral tissue with diseased, cancerous or abnormal oral tissues as well as to research new treatments. It is contemplated that the data analyzed by the system may be used to apply a fingerprint or otherwise define a normal or diseased oral tissue site. Details of the analysis of these data characteristics by the computer to identify or classify the oral tissue will now be discussed.

Upon receiving the Raman signals from probe 110, the system 100 can be configured to remove a background count from all RS spectra. In one embodiment, the background count can is determined by taking the RS spectra of the oral tissue without the laser being turned on or with the laser operating at a lower percentage of its energy output. In one embodiment, the system 100 may apply a software or hardware based smoothing technique to each RS spectrum to remove the background fluorescence signal.

In one embodiment, the system 100, and in particular the computer system 170, can calibrates each RS spectrum to the response of the probe 100 and normalize the results to an area under a Raman curve within a desired wavenumber range. In one embodiment, the computer system 170 can use a software program to analyze the normalized data. The system 100 can centers the RS spectra for each sample about its mean and scales the spectrum by its standard deviation.

The system 100, for example using software in the computer system 170, can calculate one or more sets of principal components (PCs) of the RS spectrum of the received Raman signal(s) for the tissue being examined. The system 100, for example using software in the computer system 170, can look for statistical differences between RS spectra by applying a two sided t-test on the PC to determine which PCs are significantly different from one another. Once the PCs are identified by the system 100 from the t-test, the system 100, for example using software in the computer system 170, can apply a probability calculation to the PCs to classify the samples. In one embodiment, the system 100, for example using software in the computer system 170, can apply a linear discriminate analysis, preferably with cross validation to the PCs. Additionally or alternatively, the system 100, for example using software in the computer system 170, can apply a Principle Components Analysis (PCA) to the PCs. Additionally or alternatively, the system 100, for example using software in the computer system 170, can apply a Factor Analysis to the PCs.

Based on the probability analysis, the system 100, for example using software in the computer system 170, can, in relative accurateness, identify or characterize the tissue as being normal, abnormal, diseased, or cancerous. This can be done from the results of the probability analysis alone, or by comparing the sampled tissue with data characteristics of already sampled tissue of the same person or other persons.

More details of the system and method are described below in context of a study performed using the system. In the study, Asian and Caucasian (male and female) were tested in which seven (7) oral tissue sites were sampled in vivo using the system. It should be noted that although certain values, thresholds and percentages are used to perform the study, this disclosure is not limited to those stated.

In the study, a system according to the invention was used analyze tissue emissions. The intensity of the dispersed light was measured with a NIR-optimized back illuminated, deep depletion, and liquid nitrogen cooled CCD array. A specially designed probe was made of one, ultra low OH, 200 μm diameter excitation fiber surrounded by 27, ultra low OH, 100 μm diameter collection fibers bundled together in a round configuration approximately 1.8 mm in diameter and 0.75 m long. The two stages of optical filtering were facilitated by incorporating laser line and long pass filters both at the proximal and distal ends of the probe. Control of the system was implemented by a personal computer using a custom designed program that triggered data acquisition and removed the autofluorescence background in real-time. The computer displayed graphical images of the results on a display.

In one embodiment, the RS spectra were calibrated for the spectral sensitivity of the system using a standard halogen calibration lamp (RS-10, Gamma Scientific, San Diego, Calif.) and an integrating sphere (Newport Corp. Stratford, Conn.). Briefly the enhancements included a very sensitive CCD and a very efficient (low light loss) spectrometer. Filters and fibers were also used that allow light to pass through with low loss and the generation of minimal intrinsic fluorescence. Furthermore, a parabolic array was used that allows all the light at a particular wavenumber that is collected from the sample to be projected onto the CCD in a straight line thus improving the signal to noise ratio. Together these enhancements obtained a good signal within 1 second at a preferred wavenumber range of 2700-3100 cm⁻¹

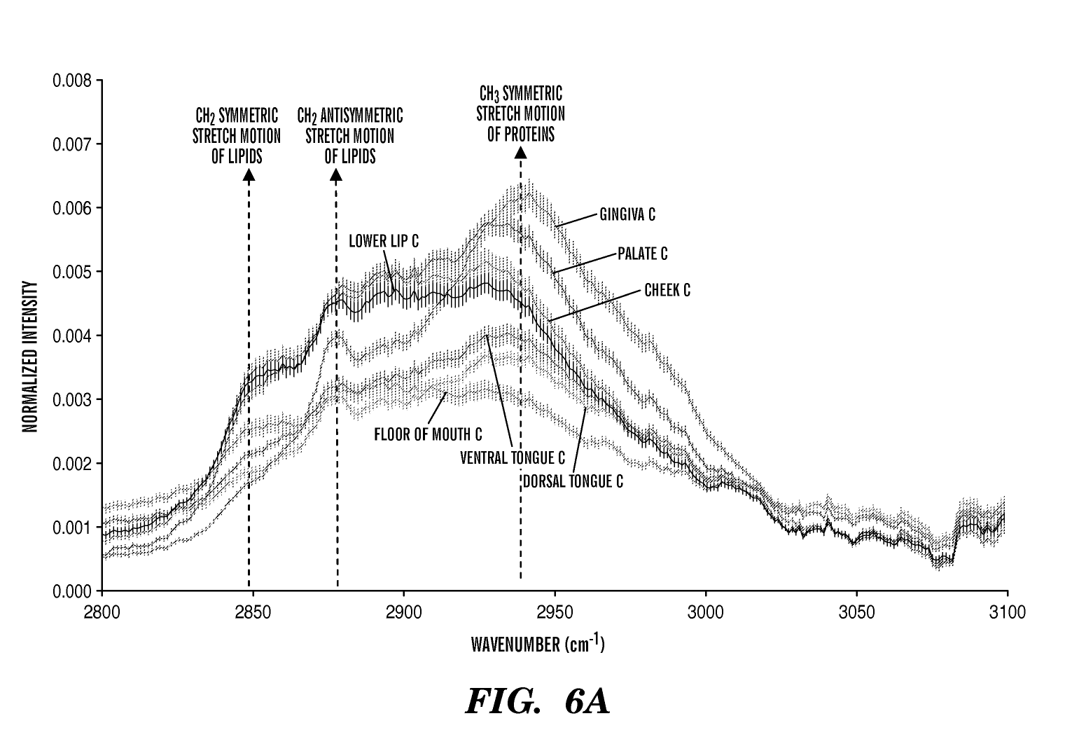

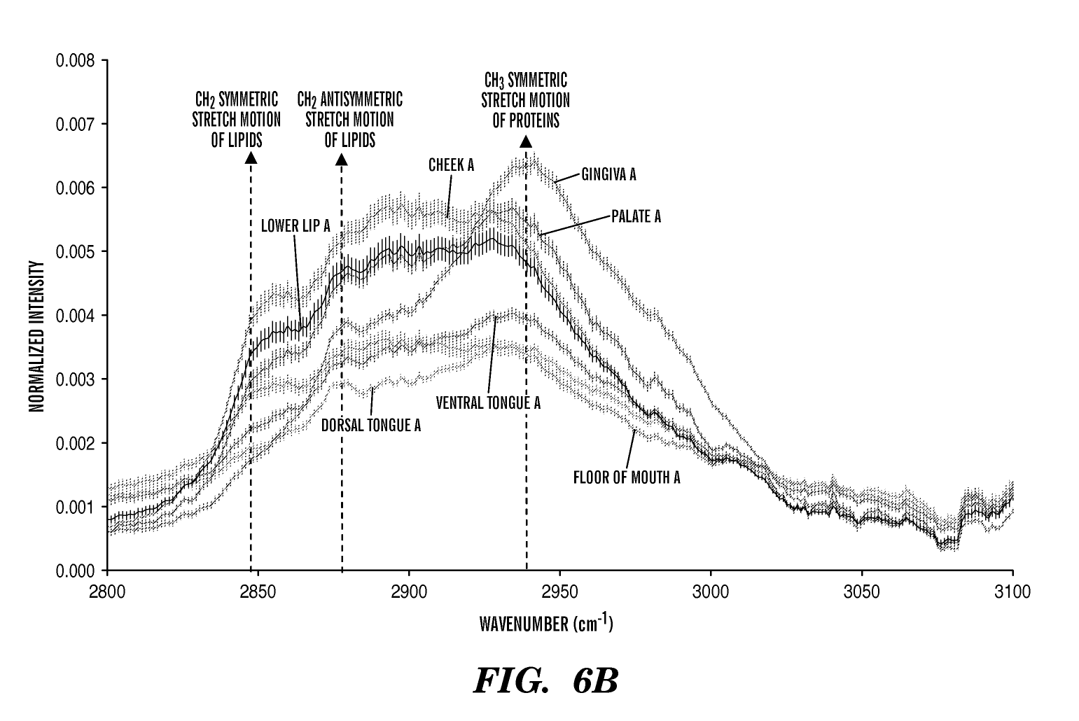

For the RS spectra being recorded, the system removed a 1 second background count from all spectra, whereby the background was obtained with the same experimental set-up as used for taking subject tissue spectra except that the red laser was not operating. The system then applied a 3 adjacent point smoothing technique to each spectrum, whereby an improved modified polynomial fitting routine using a 7th order polynomial was applied to subtract the background fluorescence signal. Each spectrum was then calibrated to the response of the instrument, and normalized to the area under the Raman curve from 1500 to 3100 cm⁻¹. The resulting spectra were grouped together by oral site and race as follows: i) all spectra, ii) Asian spectra, iii) Caucasian spectra, and (iv-x) 7 groups for the different oral sites. Such sites were movable buccal mucosa, attached gingiva, dorsal surface of the tongue, ventral surface of the tongue, the floor of the mouth, the movable mucosa of the lower lip, and the hard palate. The average results of some of these groups are shown in FIGS. 5, 6A and 6B.

The normalized data were analyzed using STATISTICA 6.0 (StatSoft Inc., Tulsa, Okla.). Prior to any analysis, 10 obvious spectral outliers (out of 351 spectra with not more than 2 spectra from each site) were rejected by inspection. The remaining spectra in each group were then centered about their mean and scaled by their standard deviation. Several sets of principal components (PCs) were calculated for each of the groups (i-x). Several sets were needed because the software was limited to 1000 data points per case whereas our spectra contained 1340 data points. To look for statistical differences between Asian and Caucasian spectra a two sided t-test was used on the PCs derived from the spectra in groups i, and iv-x, to find which PCs were significantly different; only PCs were used that accounted for 0.1% or more in the variance. Once the PCs were identified by the t-test, a linear discriminate analyzes (LDA) with cross validation was used on them to classify each spectrum as either Asian (A) or Caucasian (C). For a random classification the probability that a spectrum would be either A or C is 0.5. To avoid uncertain prediction a threshold was set for the predictive model at 0.7 that is a spectrum had to have a probability of 0.7 or greater to be classified as either A or C. If the probability was less than 0.7 (e.g., 0.6 A and 0.4 C) the spectrum was unclassified. It was determined that the best results were obtained using the spectra range 2800 to 3100 cm⁻¹. A similar procedure was used on spectra from these same groups to look for gender differences.

To determine if there were significant differences between RS spectra from different oral sites within the same ethnic group (groups ii, and iii), additional analyzes were done. The procedure was the same as that described above, except there were 7 possibilities to assign spectra (e.g. 7 oral tissue sites being examined). The random assignment probability was therefore 1/7 or 0.143. To avoid uncertain prediction a threshold for the predictive model was set at 0.50 (that is a spectrum had to have a probability >0.50 to be classified). Although this threshold is lower than that used to separate Asian/Caucasian and male/female spectra, 0.50 is 0.357 above random and as such spectra meeting this criterion will be significantly different from the average spectra of other sites. Furthermore a >0.50 threshold stops any spectrum being classified as belonging to two or more oral sites which will complicate the interpretation of the results. The best results were obtained using the spectral range from 2800 to 3100 cm⁻¹ rather than the entire range.

The average spectra from different oral sites in the 1500-3100 cm⁻¹ range is shown in FIG. 5. All spectra contained a large peak near 1665 cm⁻¹ (FIG. 5), which was most likely the Raman peak due to amide I vibrations with some contributions from the C═C stretching motion of lipids, and H₂O bending motions. The broad peak centered on 3000 cm⁻¹ was clearly the well known Raman peak due to a combination of lipids and proteins. Low intensity broad emissions that extended from 2000 to 2300 cm⁻¹ in all spectra were probably made up of H₂O molecule librations and various carbon/nitrogen/oxygen modes. Above 3100 cm⁻¹ there was some evidence in the raw data for a Raman peak around 3300 cm⁻¹ (not shown). This was due to OH stretching motions of water molecules.

Each of the scanned oral sites displayed distinct spectra (FIGS. 6A and 6B). The spectra from some sites were on average statistically different from other sites—the error bars shown are the calculated errors on the means. 68% of new average spectra would lay within the error bars, and 95% would lay within error bars twice as large and 99.7% would lie within error bars 3 times as large. Spectra obtained from the lower lip and cheek were similar and tended to peak at 2850, 2900 and 2925 cm⁻¹. In contrast, gingival spectra peaks were noted at 2880 and 2940 cm⁻¹. Similarly, maximal intensity spectra of 2875 and 2930 cm⁻¹ were noted for the hard palate. The ventral and dorsal tongue spectra appeared somewhat similar on visual inspection with peaks at 2870 and 2935 cm⁻¹. The floor of the mouth was different than the other tissues and displays a rather shallow climb and a broader range of peaks including 2850, 2890, and 2930 cm⁻¹.

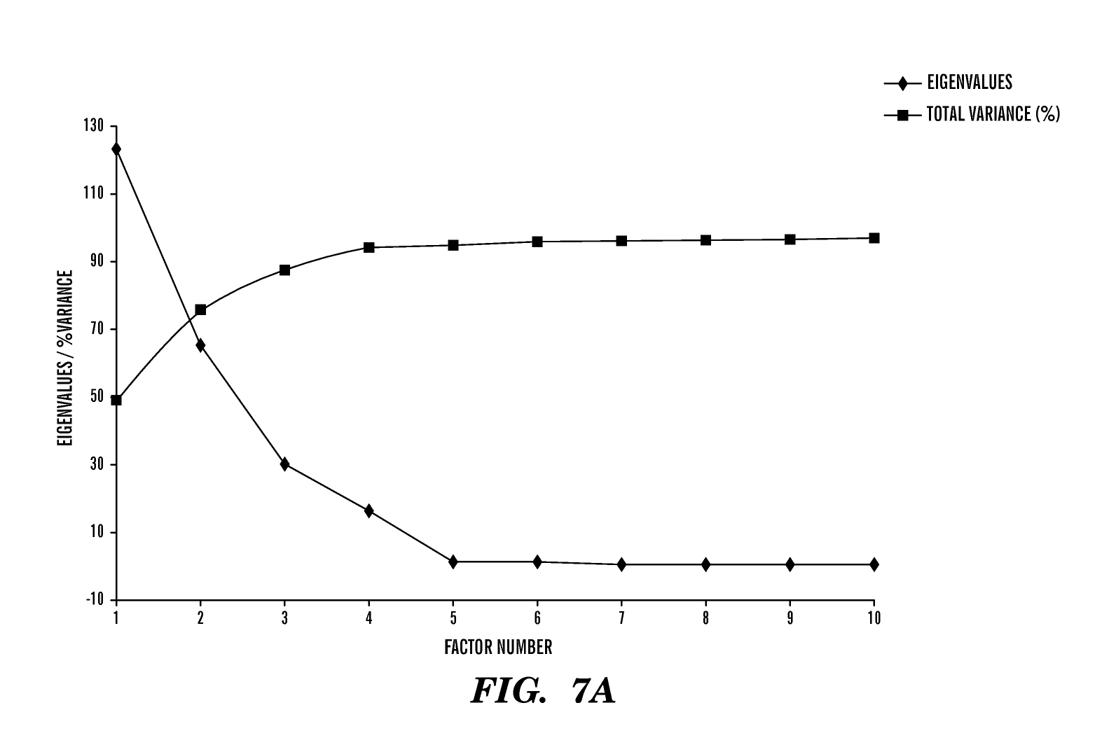

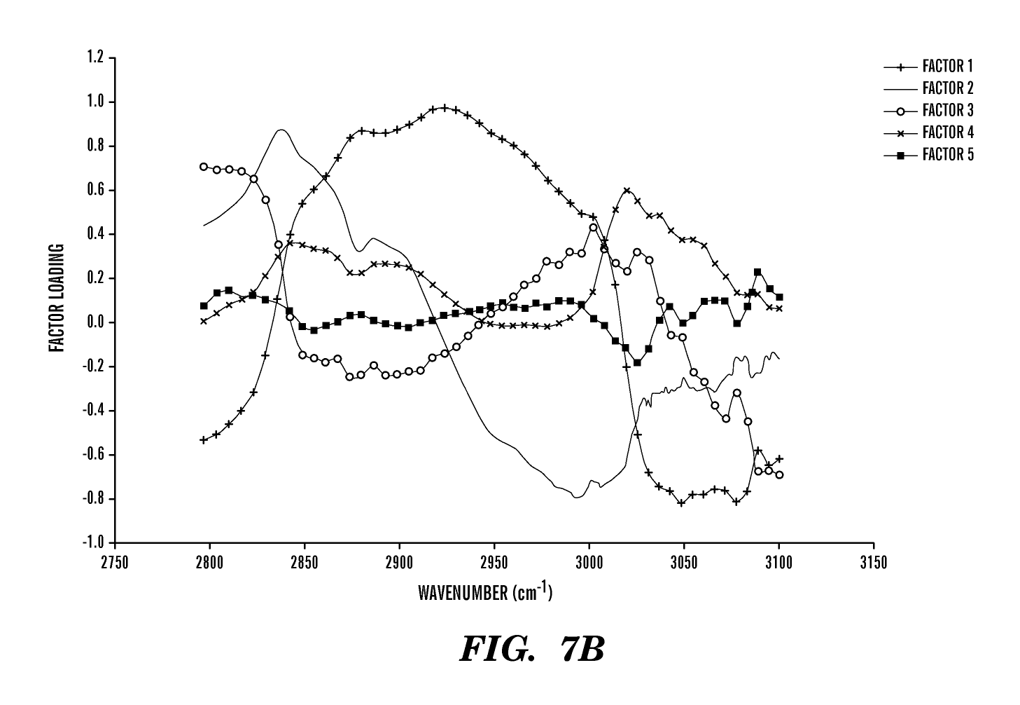

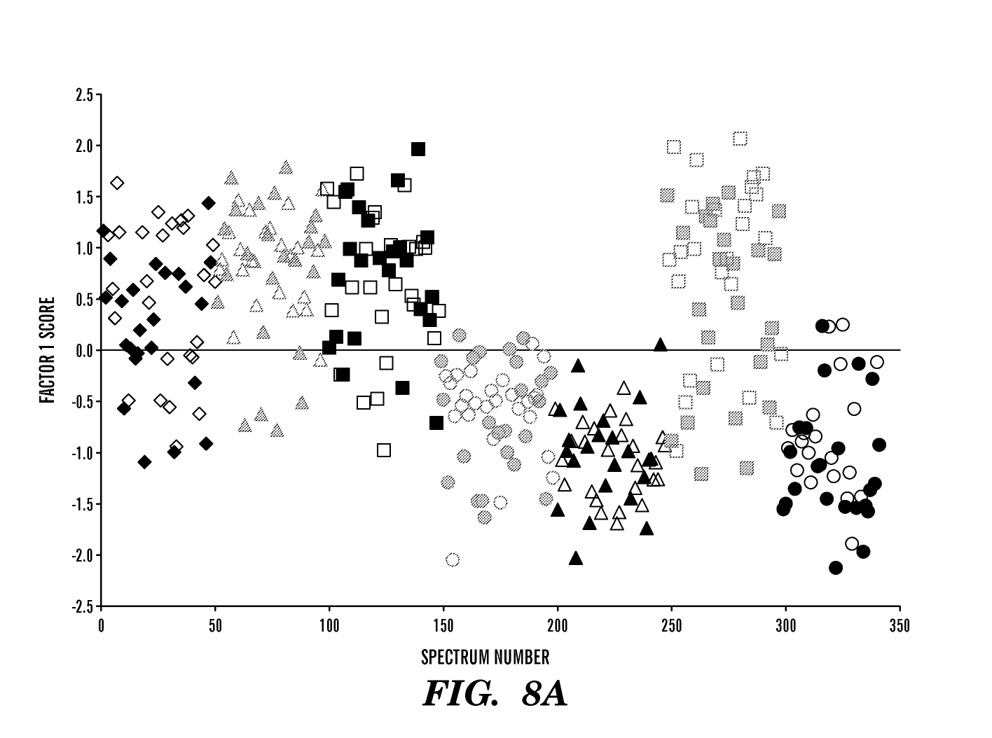

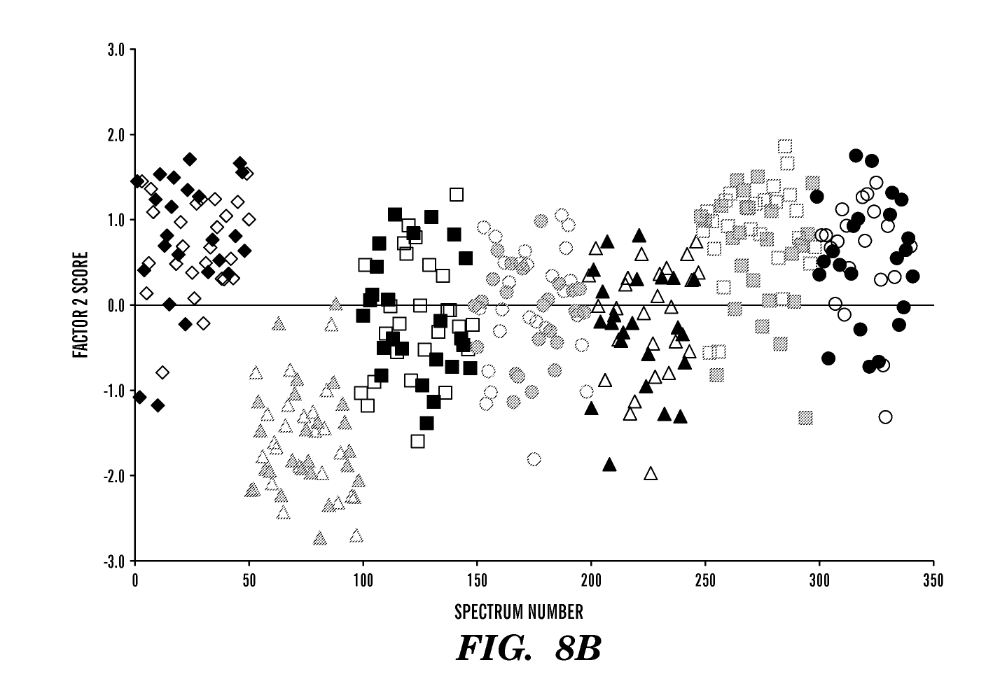

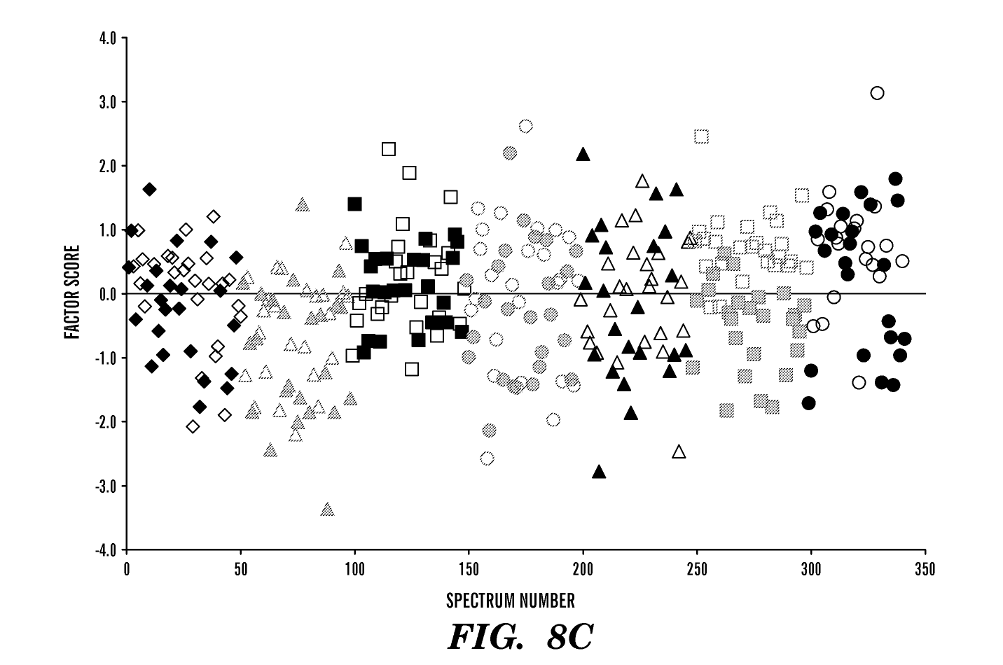

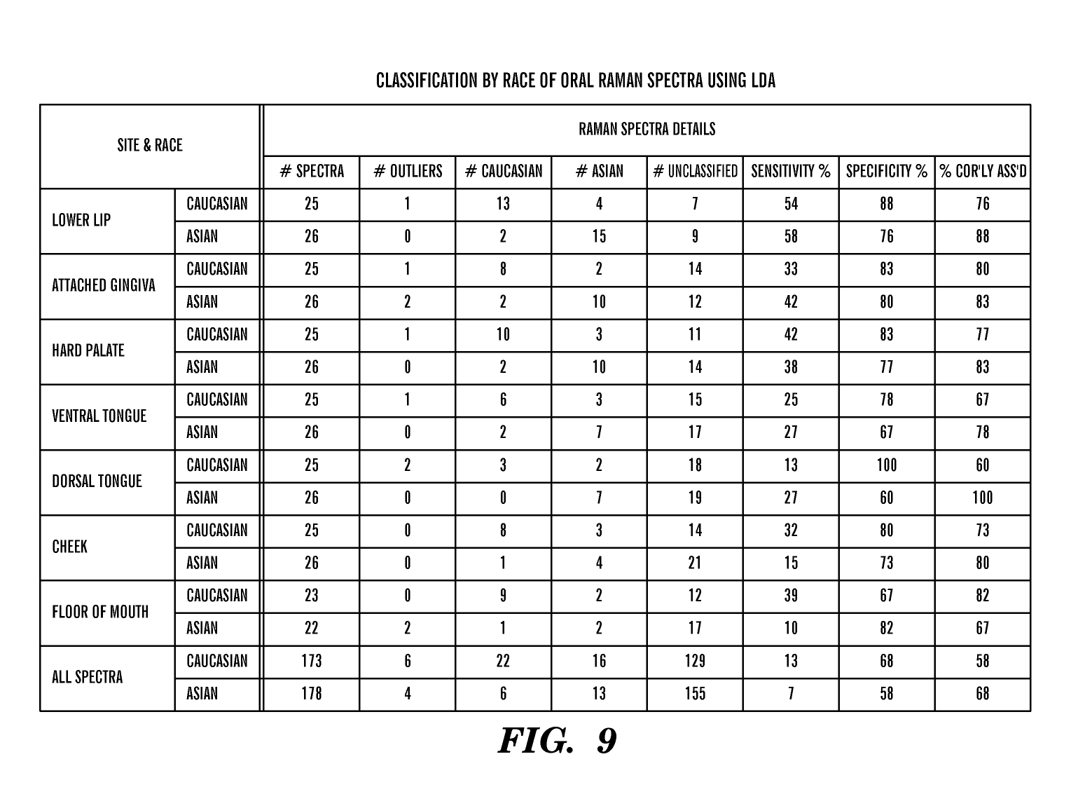

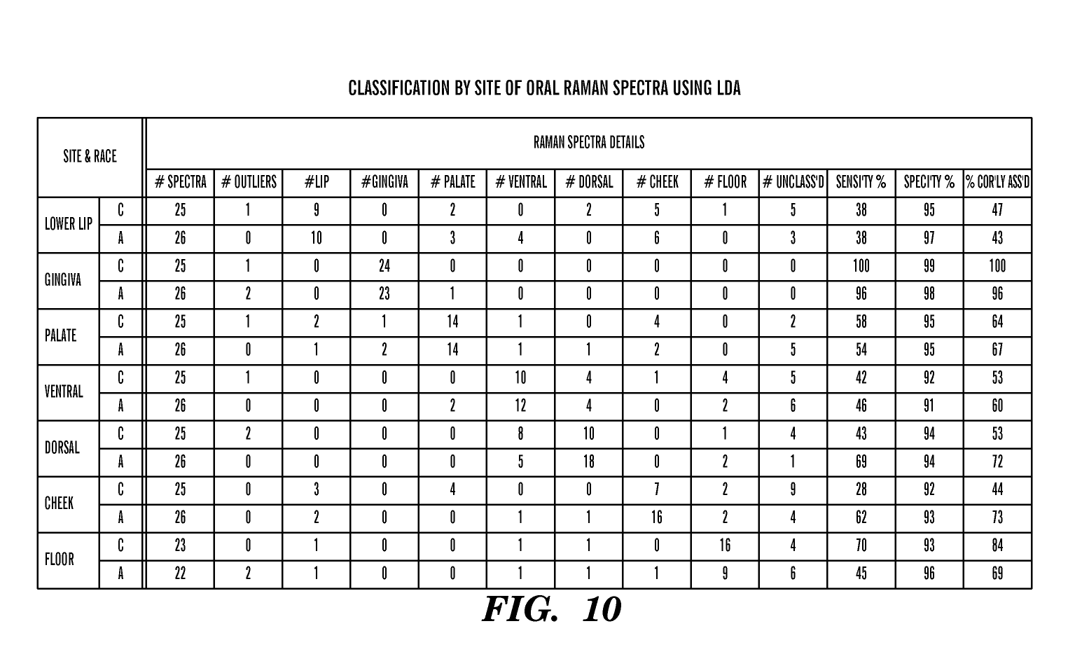

In performing a PCA analysis on the RS spectra, the Eigenvalues for the PCA of all the spectra (group i) dropped rapidly to low levels after about 5 factors (FIG. 7A), and these factors accounted for over 95% of total variance. The loading plots for the first 5 factors are shown in FIG. 7B. T-tests on the first 10 factors identified 2 or more with significant p-values (<0.05) for discriminating between spectra from two oral sites. The most significant factors for nearly all sites was either factor 1 (p<2×10⁻⁵) or factor 2 (p<3×10⁻⁵). The exception to this was the comparison between lower lip and cheek spectra where factor 4 (p=0.001) was the most significant. FIGS. 8A-8C show scatter plots of factors 1, 2 and 4 respectively. The LDA on all the significant factor scores by race and site is outlined in Tables 1 and 2.

From the study, the RS spectra clearly show the Raman peaks due to proteins, lipids and water. The undesirable noise in the spectra was small compared to the variation in Raman peak intensities. The polynomial fitting to remove the fluorescence was carried out before spectral intensity calibration and this was found to produce the best fit to the data. The 2800-3100 cm⁻¹ range analyzed seemed largely free of any significant artifacts, and showed clear differences in average Raman intensity for different groupings.

Where LDA was used, the classification of spectra was nearly 100% correct in some cases, but in others, only 62% were correct. The correct classification percentage goes up if one increases the probability threshold. Surprisingly the LDA could correctly classify a significant fraction of the spectra from each site by race using a 0.7 threshold even though the average spectra showed little difference. This occurred because the LDA were based on PCs that only accounted for small percentages of the total variance.

The study supports applying RS technology to the diagnosis of oral mucosal pathology by defining the spectral signal for specific mucosal sites within the mouth. It was demonstrated that the RS signal was consistent among subjects of different ethnicities and gender, and that the extent of the signal was dependent on the type of oral mucosa being evaluated. These data thus provide the baseline against which abnormal mucosal changes can be defined. Signals varied between some tissues (gingiva and cheek) and similar with others (dorsal and ventral tongue) primarily due to the extent of the differences in the molecular structure. Tissues composed of similar relative amounts of lipids, carbohydrates and proteins, will resemble each other to a greater degree than those that are not. Future studies will involve identification of the molecular structures that will enhance understanding of not only tissue types but differences amongst races.

Various methods of non-invasive tissue diagnosis have been studied in the head and neck region. More recently autofluorescence techniques have been studied. In the oral cavity, sensitivity, and specificity values of 88% and 100%, respectively, have been reported in distinguishing neoplasia from normal tissue. For the larynx, similar diagnostic sensitivity has been reported but the specificity for distinguishing malignant from benign lesions may be as low as 50%. RS has a potential advantage over these techniques in that it can provide a molecular fingerprint of tissue. However the signal may be obscured by autofluorescence, which is also induced by molecular excitation. For this reason, near-infrared (NIR) wavelengths are used in preference to visible light for measuring Raman scattering in biomedical applications.

Using techniques ranging from empirical analysis of individual peaks to multivariate analysis of multiple spectral peaks, a number of in vitro and in vivo studies have reported sensitivity and specificity values of over 90% for distinguishing cancer from normal tissue using RS. In the oral cavity, the use of RS to achieve a non-invasive real time optical diagnosis has the potential to provide an adjunct to visual oral examination. Examples where non-invasive identification of pathology may be of particular value include surveillance of conditions such as inflammatory, autoimmune diseases and dysplasia.

Accordingly, from the study, in vivo Raman spectra from the oral cavity were successfully acquired. In vivo Raman spectra taken from the oral cavity of 51 human subjects did not show strong differences between Asian and Caucasian subgroups. However the spectra for different oral sites within the same ethnic group were significantly different and clearly separable.

What is meant by “mucosal tissues” are tissues that are composed in part of cells of mesenchymal and epithelial origin. Examples of mucosal tissues include, but are not limited to, vaginal, oral, corneal and rectal.

While embodiments and applications have been shown and described, it would be apparent to those skilled in the art having the benefit of this disclosure that many more modifications than mentioned above are possible without departing from the inventive concepts disclosed herein. The invention, therefore, is not to be restricted except in the spirit of the appended claims.

Other embodiments are within the scope and spirit of the invention. For example, due to the nature of software, functions described above can be implemented using software, hardware, firmware, hardwiring, or combinations of any of these. Features implementing functions may also be physically located at various positions, including being distributed such that portions of functions are implemented at different physical locations.

Further, while the description above refers to the invention, the description may include more than one invention.

What is claimed is:

1-58. (canceled)

59. A method for characterizing mucosal tissue using Raman Spectroscopy (RS) comprising:
emitting an illumination light from a light source of a medical device directed toward mucosal tissue;
receiving a Raman signal from the mucosal tissue in response to being induced by the illumination light, the Raman signal including data characteristics recorded by a computer coupled to the medical device;
performing a statistical probability analysis on at least a portion of the data characteristics to characterize mucosal tissue.

60. The method of claim 59 further comprising:
identifying at least one RS spectrum ef in the stored data characteristics;
calculating a principal component value for the at least one RS spectrum;
comparing the data characteristics of the mucosal tissue with already stored data characteristics of a healthy mucosal tissue similar to the diagnosed mucosal tissue;
identifying at least two principal component values from each of the diagnosed mucosal tissue and the healthy mucosal tissue, the at least two principal component values from the diagnosed mucosal tissue being statistically different from the at least two principal component values from the healthy mucosal tissue within a predetermined percentage;
performing a probability analysis on the identified principal component values, wherein the probability analysis is based on a predetermined threshold value; and
identifying the mucosal tissue based on the probability analysis.

61. (canceled)

62. The method of claim 59, further comprising removing a background count from the each of the RS spectrum.

63-65. (canceled)

66. The method of claim 60, wherein identifying the at least two principal component values that are statistically different includes performing a t-test on the at least two principal component values from the diagnosed mucosal tissue and the at least two principal component values from the healthy mucosal tissue.

67. The method of claim 59, wherein the probability analysis further comprises at least one of, a linear discriminate analysis, a principle components analysis and a factor analysis.

68-72. (canceled)

73. The method of claim 59, wherein the characterization of the mucosal tissue further comprises characterizing the mucosal tissue as being in one of, a healthy state, an abnormal state and a diseased state.

74-78. (canceled)

79. A Raman spectrograph probe for measuring Raman spectra of tissue, the probe comprising:
an elongated handle extending from a first end to a second end and a tissue contacting tip extending a predefined distance, f, from the first end and adapted to position the first end the predefined distance, f, from tissue in contact with the tissue contacting tip;
a first excitation fiber extending from the first end, through at least a portion of the handle to a distal end adapted to be connected to a first excitation laser source whereby first excitation laser radiation can be transferred to through the first excitation fiber and projected through the first end; and
at least one emission fiber extending from the first end, through at least a portion of the handle to a distal end adapted to be connected to a Raman spectrograph whereby Raman spectra received at the first end can be transferred through at least one emission fiber to the Raman spectrograph.

80. A Raman spectrograph probe according to claim 79 wherein the tissue contacting tip is removable.

81. A Raman spectrograph probe according to claim 79 wherein the tissue contacting tip includes an opening allowing laser radiation from the first excitation fiber to be transmitted through the opening and Raman spectra to be transferred through the opening to at least one emission fiber, the contact tip being opaque to block radiation from being transmitted through the contact tip into the opening.

82. A Raman spectrograph probe according to claim 79 wherein the tissue contacting tip includes an opening allowing laser radiation from the first excitation fiber to be transmitted through the opening and Raman spectra to be transferred through the opening to the emission fibers, the opening in the contact tip defining an inner surface and the inner surface including a material adapted to absorb substantially all radiation incident on the inner surface.

83-84. (canceled)

85. A Raman spectrograph probe according to claim 82 wherein at least the inner surface of the tissue contacting tip is formed of a material having low Raman spectra emission properties in response to the first excitation laser radiation.

86. A Raman spectrograph probe according to claim 82 wherein the contact tip is adapted to contact tissue to be studied and position the first end of the Raman spectrograph probe a predefined distance in the range from 3 to 10 mm from the tissue.

87-89. (canceled)

90. A Raman spectrograph probe according to claim 79 further comprising a band pass filter positioned adjacent the first excitation fiber at the first end, the band pass filter being adapted to pass only radiation in a predefined wavelength band that includes the first wavelength band.

91. A Raman spectrograph probe according to claim 90 wherein the band pass filter is adapted to pass radiation having a wavelength in the range of 700 to 850 nanometers.

92. A Raman spectrograph probe according to claim 1 further comprising a first excitation laser source connected to the first excitation fiber and a Raman spectrograph connected to at least one emission fiber.
